(19) United States
(12) Patent Application Publication
(10) Pub. No.: US 2013/0281871 A1
(43) Pub. Date: Oct. 24, 2013

(54) SYSTEM AND METHOD FOR CLASSIFYING THE RESPIRATORY HEALTH STATUS OF AN ANIMAL

(71) Applicants: Brad J. White, Manhattan, KS (US); Dan R. Goehl, LaGrange, MO (US)

(72) Inventors: Brad J. White, Manhattan, KS (US); Dan R. Goehl, LaGrange, MO (US)

(73) Assignee: PROFESSIONAL BEEF SERVICES, LLC, Canton, MO (US)

(21) Appl. No.: 13/449,671

(22) Filed: Apr. 18, 2012

(51) Int. Cl. A61B 5/11; A61B 5/08

(52) U.S. Cl. 600/529

(57) ABSTRACT

Systems and methods are provided for determining the respiratory health status of an animal. The systems and methods utilize location data of individual animals to generate variables describing the behavior of the individual animals. The systems and methods evaluate the variables to assess the health status of individual animals.

### FIELD

The present invention generally relates to systems and methods for classifying the respiratory health status of an animal, and more particularly, to classifying the respiratory health status of an animal based on positional data gathered on the animal.

### BACKGROUND

Bovine respiratory disease (BRD) is the most common and costly ailment of beef cattle after weaning, which typically occurs at approximately seven months of age. In addition to the costs associated with performance loss and/or death of afflicted animals, the cattle industry spends millions of dollars each year attempting to prevent and treat BRD. One of the shortfalls in mitigating the negative impact of BRD is the inability to rapidly and accurately diagnose afflicted cattle.

The most common system and method used to identify cattle, particularly calves, with BRD is the visual appraisal of individual animals. Visual observation has been shown to be relatively inaccurate. For example, according to at least one source, the current system of visual observation may be effective at detecting only 65% of afflicted animals. See White et al., Bayesian estimation of the performance of using clinical observations and harvest lung lesions for diagnosing bovine respiratory disease in post-weaned beef calves, 21 J. Vet. Diagn. Invest. 1, 446-53 (2009). The inability to correctly identify sick calves not only limits the potential efficacy of antimicrobial medications, but also places a strain on limited labor resources. The inability to correctly identify healthy calves may result in unnecessary antimicrobial medication use and labor costs. Despite its inadequacies, no efficient system or method has been created to replace visual observation.

Although visual assessment is most common, there are a number of existing methods for diagnosing BRD. Many of these methods are based on physiologic indices measured prior to initiation of a clinical disease. However, measuring physiologic indices including temperature, respiratory rate, heart rate, and many blood parameters prior to initiation of a clinical disease does not adequately discriminate the wellness state of calves with respiratory disease. See Hanzlicek et al, Serial evaluation of physiologic, pathological, and behavioral changes related to disease progression of experimentally induced Mannheimia haemolytica pneumonia in post-weaned calves, 71 Am. J. Vet. Res. 249, 359-69 (2010).

A few methods focus on improving disease diagnosis at the time of initial disease onset. For example, U.S. Patent Publication No. 2009/0137918 ('918 publication) describes how monitoring respiratory character may be predictive of disease state. However, the monitoring discussed in the '918 publication can be performed only after an individual animal is identified and retrieved from a housing area for further evaluation. Therefore, if an animal is not identified as potentially having respiratory disease, a diagnosis cannot be made unless all animals are evaluated. U.S. Pat. No. 7,931,593 ('593 patent) evaluates several components of the animal physiologic state and growth patterns. However, similar to the '918 publication, the evaluation discussed in the '593 patent requires all animals to be evaluated individually in a confined area, requiring movement from the housing area. Thus, a need exists for systems and methods which can remotely evaluate and determine the respiratory health status of an animal without requiring movement from the housing area.

Previous research also documents potential changes in the location of disease-stricken calves. See Sowell et al., Radio frequency technology to measure feeding behavior and health of feedlot steers, 59 Appl. Anim. Behav. Sci. 253, 277-284 (1998). U.S. Pat. Nos. 6,375,612 and 6,569,092 utilize location data and proximity to defined areas data to predict health status. However, neither of these systems account for time dependent behavioral trends including the actual level of animal activity and the social interactions with all other animals within the housing area.

Despite the number of existing methods that evaluate a wide variety of animal data parameters, there is still a need for improved systems and methods that accurately and timely diagnose disease-stricken animals.

### SUMMARY

In accordance with the present invention, systems and methods are provided that improve the accuracy and timeliness of disease detection through evaluation of quantitative measures. To advantageously influence therapeutic decisions, embodiments of the present invention collect information at opportune times, accurately analyze the information, and timely report the results to facilitate effective treatment of diseased animals. The accurate, timely diagnosis of diseased animals is beneficial to both the animals and the production industry. Improvement in disease identification accuracy and speed impacts several areas of beef production including appropriate antimicrobial use, animal welfare, and improved production (labor) efficiency.

The systems and methods utilize time-series positional data of individual animals to generate variables describing the behavior of the individual animals. The positional data may be collected by a variety of devices known in the art as long as the positional data is attributable to an individual animal, a specific point in time, and at least a two-dimensional location within the area in which the animal is located. The positional data is used to create variables, which in turn are used to establish the respiratory health status of individual animals.

The variables include at least one activity variable, at least one proximity variable, and/or at least one social variable. The at least one activity variable describes the movement of individual animals. The at least one proximity variable describes the proximity of individual animals to an area of interest. The at least one social variable describes the social interactions of individual animals with other animals. The at least one activity variable, at least one proximity variable, and/or at least one social variable may be aggregated over a predetermined time period, such as an hour. The biological progression of illness is complex, and individual animals do not express illness in the same manner. Thus, activity, proximity, and social variables can be utilized to identify health patterns that are unrecognized without inclusion of all of the variables. For example, a decreased overall activity rate may indicate illness or health depending on the number of social interactions with other animals in the pen. Additionally, an increased activity rate may indicate illness when the animal spends less time in the proximity of the feeding area, but may indicate health if the animal spends more time in the feeding area. The inclusion of at least three categories of variables provides a more accurate classification of the wellness state of the animal. These variables, as defined by their respective data, are used in conjunction with at least one time-series behavioral trend variable to classify the respiratory health status of individual animals.

At least one behavioral trend variable is created based on at least one activity variable, at least one proximity variable, and/or at least one social variable associated with an individual animal. By analyzing the behavioral trends of individual animals over a set period of time, the classification system may accurately depict potentially meaningful changes in behavior. The at least one behavioral trend variable may comprise a moving average variable, an upper control limit variable, a lower control limit variable, a relative strength index variable, and a behavioral channel index variable. The resulting set of behavioral time series trend data are used in connection with at least one dynamic disease detection algorithm to classify the respiratory health status of individual animals.

A diagnostic algorithm is used to classify the respiratory health status of individual animals. The diagnostic algorithm receives at least one activity variable, at least one proximity variable, at least one social variable, and/or at least one behavioral trend variable associated with an individual animal as inputs and outputs a respiratory health status of an individual animal. As individual animals may display distinct behavioral patterns when expressing signs of clinical illness, a variety of dynamic diagnostic algorithms may be applied to the behavioral trend data. The algorithm may include an artificial neural network algorithm, such as a backpropagation algorithm, a decision tree learning algorithm, a Bayes algorithm, a logistic regression algorithm, or any combination thereof. In one embodiment, a decision tree algorithm, a naïve Bayesian classification algorithm, a neural network algorithm, and a logistic regression algorithm are applied to the behavioral trend data. Historical behavioral and respiratory health data of animals having known respiratory health statuses are used to create the diagnostic algorithm.

A report is generated that details the respiratory health status of an individual animal over a designated time period. The report may be in the form of, for example, at least one of a user interface, a printed report, a text message, or an email message. The designated time period may be a 12-hour period, and the report may include the health status of the individual animal for the current 12-hour period and at least one previous 12-hour period. The results of the classification systems and methods can be used to determine the respiratory health status of individual animals, thereby influencing preventative health and therapeutic decisions. In one embodiment, the health status of a calf is assessed.

The invention disclosed herein enables the detection of BRD at the time of initial disease onset. Additionally, the invention can remotely evaluate individual animals and determine their respiratory health status without requiring removal of the animals from the housing area. Further, the invention may utilize a plurality of dynamic disease detection algorithms to more accurately diagnose diseased animals.

The term “a” or “an” entity, as used herein, refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium”, as used herein, refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

The terms “determine”, “calculate” and “compute,” and variations thereof, as used herein, are used interchangeably and include any type of methodology, process, mathematical operation or technique.

The term “module”, as used herein, refers to any known or later developed hardware, software, firmware, artificial intelligence, fuzzy logic, or combination of hardware and software that is capable of performing the functionality associated with that element.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

The preceding is a summary of the present invention. This summary is neither an extensive nor exhaustive overview of the present invention and its various aspects, embodiments, and/or configurations. It is intended neither to identify key or critical elements of the present invention nor to delineate the scope of the present invention but to present selected concepts of the present invention in a simplified form as an introduction to the more detailed description presented below. As will be appreciated, other aspects, embodiments, and/or configurations of the present invention are possible utilizing, alone or in combination, one or more of the features set forth above or described in detail below. Other features and advantages of the present invention will become apparent from a review of the following detailed description, taken in conjunction with the drawings.

### BRIEF DESCRIPTION OF THE DRAWINGS

FIG. 1 is an example communications/data processing network system that may be used in conjunction with embodiments of the present invention;

FIG. 2 is an example computer system that may be used in conjunction with embodiments of the present invention;

FIG. 3 is a flowchart illustrating one embodiment of a respiratory health status classification method;

FIG. 4 is a block diagram of one embodiment of a behavioral trend engine utilized in generating behavioral trend variables;

FIG. 5 is a list of variables and the associated descriptions usable in embodiments of the present invention;

FIG. 6 is a block diagram of one embodiment of a classification engine utilized in classifying the respiratory health status of individual animals;

FIG. 7 is a block diagram of one embodiment of a classification engine utilized in classifying the respiratory health status of individual animals;

FIG. 8 is a flowchart illustrating one embodiment of generating a classification module;

FIG. 9 is a block diagram of one embodiment of a decision tree module;

FIG. 10 is a block diagram of one embodiment of a framework for a neural network module;

FIG. 11 is a list of variables and associated data for a logistic regression module generated from time series positional data of animals with known health status usable in embodiments of the present invention;

FIG. 12 is a sample report illustrating results for a 24-hour period from two independent animals (denoted by tag number) and the results from each of the predictive models (including the cumulative results labeled as model series); and

FIG. 13 is a sample report illustrating a respiratory health status report displaying the overall health status classification for the current 12-hour period and previous periods of time.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

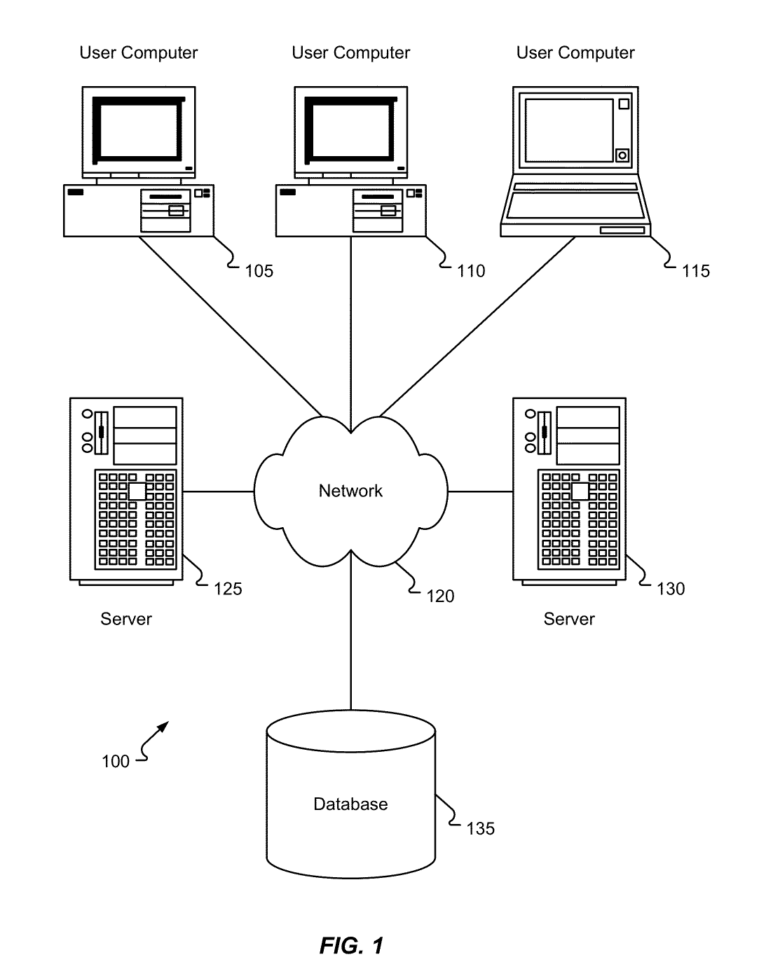

Referring to FIG. 1, an example network system is provided that may be used in connection with the classification systems and methods disclosed herein. More specifically, FIG. 1 illustrates a block diagram of a system 100 that may use a web service connector to integrate an application with a web service. The system 100 includes one or more user computers 105, 110, and 115. The user computers 105, 110, and 115 may be general purpose personal computers (including, merely by way of example, personal computers and/or laptop computers running various versions of Microsoft Corp.'s Windows™ and/or Apple Corp.'s Macintosh™ operating systems) and/or workstation computers running any of a variety of commercially-available UNIX™ or UNIX-like operating systems. These user computers 105, 110, 115 may also have any of a variety of applications, including for example, database client and/or server applications, and web browser applications. Alternatively, the user computers 105, 110, and 115 may be any other electronic device, such as a thin-client computer, Internet-enabled mobile telephone, and/or personal digital assistant, capable of communicating via a network (e.g., the network 120 described below) and/or displaying and navigating web pages or other types of electronic documents. Although the exemplary system 100 is shown with three user computers, any number of user computers may be supported.

System 100 further includes a network 120. The network 120 may be any type of network familiar to those skilled in the art that can support data communications using any of a variety of commercially-available protocols, including without limitation TCP/IP, SNA, IPX, AppleTalk, and the like. Merely by way of example, the network 120 maybe a local area network (“LAN”), such as an Ethernet network, a Token-Ring network and/or the like; a wide-area network; a virtual network, including without limitation a virtual private network (“VPN”); the Internet; an intranet; an extranet; a public switched telephone network (“PSTN”); an infra-red network; a wireless network (e.g., a network operating under any of the IEEE 802.11 suite of protocols, the Bluetooth™ protocol known in the art, and/or any other wireless protocol); and/or any combination of these and/or other networks.

The system 100 may also include one or more server computers 125, 130. One server may be a web server 125, which may be used to process requests for web pages or other electronic documents from user computers 105, 110, and 120. The web server can be running an operating system including any of those discussed above, as well as any commercially-available server operating systems. The web server 125 can also run a variety of server applications, including HTTP servers, FTP servers, CGI servers, database servers, Java servers, and the like. In some instances, the web server 125 may publish operations available as one or more web services.

The system 100 may also include one or more file and/or application servers 130, which can, in addition to an operating system, include one or more applications accessible by a client running on one or more of the user computers 105, 110, 115. The server(s) 130 may be one or more general purpose computers capable of executing programs or scripts in response to the user computers 105, 110 and 115. As one example, the server may execute one or more web applications. The web application may be implemented as one or more scripts or programs written in any programming language, such as Java™, C, C#™ or C++, and/or any scripting language, such as Perl, Python, or TCL, as well as combinations of any programming/scripting languages. The application server(s) 130 may also include database servers, including without limitation those commercially available from Oracle, Microsoft, Sybase™, IBM™ and the like, which can process requests from database clients running on a user computer 105.

In some embodiments, an application server 130 may create web pages dynamically for displaying the development system. The web pages created by the web application server 130 may be forwarded to a user computer 105 via a web server 125. Similarly, the web server 125 may be able to receive web page requests, web services invocations, and/or input data from a user computer 105 and can forward the web page requests and/or input data to the web application server 130.

In further embodiments, the server 130 may function as a file server. Although for ease of description, FIG. 1 illustrates a separate web server 125 and file/application server 130, those skilled in the art will recognize that the functions described with respect to servers 125, 130 may be performed by a single server and/or a plurality of specialized servers, depending on implementation-specific needs and parameters.

The system 100 may also include a database 135. The database 135 may reside in a variety of locations. By way of example, database 135 may reside on a storage medium local to (and/or resident in) one or more of the computers 105, 110, 115, 125, 130. Alternatively, it may be remote from any or all of the computers 105, 110, 115, 125, 130, and in communication (e.g., via the network 120) with one or more of these. In a particular set of embodiments, the database 135 may reside in a storage-area network (“SAN”) familiar to those skilled in the art. Similarly, any necessary files for performing the functions attributed to the computers 105, 110, 115, 125, 130 may be stored locally on the respective computer and/or remotely, as appropriate. In one set of embodiments, the database 135 may be a relational database, such as Oracle 10i™, that is adapted to store, update, and retrieve data in response to SQL-formatted commands.

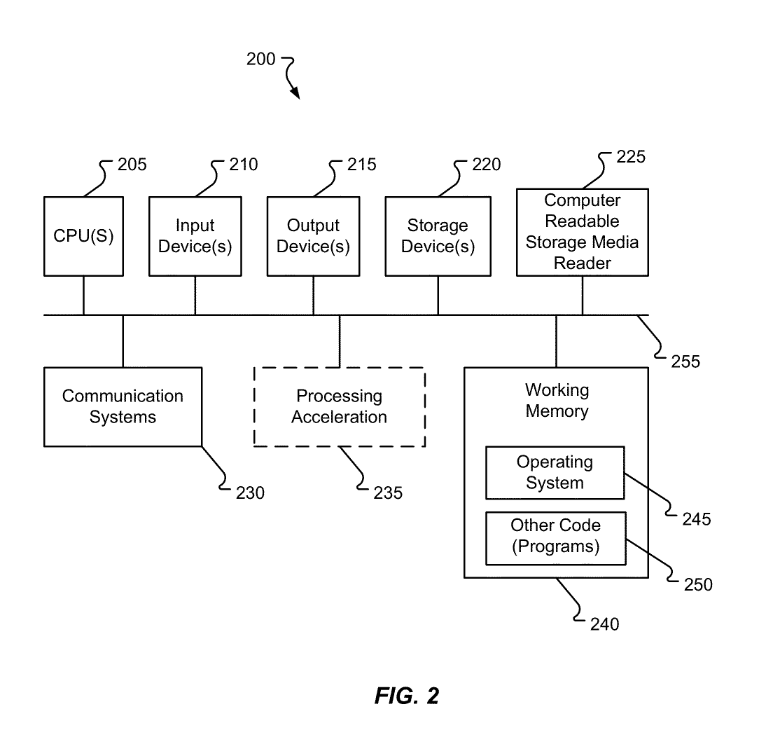

Referring to FIG. 2, an example computer system is provided that may be used in connection with the classification systems and methods disclosed herein. More specifically, FIG. 2 illustrates one embodiment of a computer system 200 upon which a web service connector or components of a web service connector may be deployed or executed. The computer system 200 is shown comprising hardware elements that may be electrically coupled via a bus 255. The hardware elements may include one or more central processing units (CPUs) 205; one or more input devices 210 (e.g., a mouse, a keyboard, etc.); and one or more output devices 215 (e.g., a display device, a printer, etc.). The computer system 200 may also include one or more storage device 220. By way of example, storage device(s) 220 may be disk drives, optical storage devices, solid-state storage device such as a random access memory (“RAM”) and/or a read-only memory (“ROM”), which can be programmable, flash-updateable and/or the like.

The computer system 200 may additionally include a computer-readable storage media reader 225; a communications system 230 (e.g., a modem, a network card (wireless or wired), an infra-red communication device, etc.); and working memory 240, which may include RAM and ROM devices as described above. In some embodiments, the computer system 200 may also include a processing acceleration unit 235, which can include a DSP, a special-purpose processor and/or the like.

The computer-readable storage media reader 225 can further be connected to a computer-readable storage medium, together (and, optionally, in combination with storage device(s) 220) comprehensively representing remote, local, fixed, and/or removable storage devices plus storage media for temporarily and/or more permanently containing computer-readable information. The communications system 230 may permit data to be exchanged with the network 220 and/or any other computer described above with respect to the system 200.

The computer system 200 may also comprise software elements, shown as being currently located within a working memory 240, including an operating system 245 and/or other code 250, such as program code implementing a web service connector or components of a web service connector. It should be appreciated that alternate embodiments of a computer system 200 may have numerous variations from that described above. For example, customized hardware might also be used and/or particular elements might be implemented in hardware, software (including portable software, such as applets), or both. Further, connection to other computing devices such as network input/output devices may be employed.

It should be appreciated that the methods described herein may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

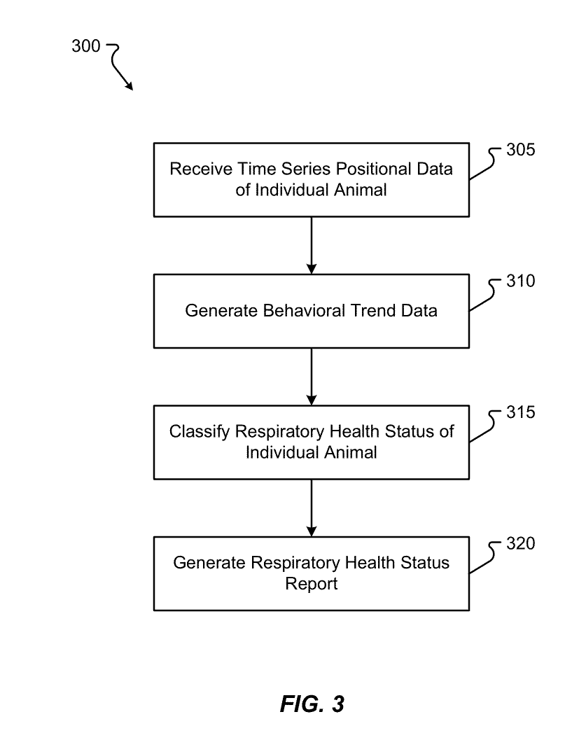

FIG. 3 depicts one embodiment of a respiratory health status classification method 300. At step 305, a classification system receives time-series positional data of individual animals. The classification system may monitor and collect time-series positional data of individual animals. For example, the classification system may identify and track the movements of individual animals within a housing area including a pen, a corral, a fenced pasture, or other confined areas known in the art. The positional data is attributable to individual animals, a specific time, and at least a two-dimensional location within the housing area. The data can be collected in a continuous manner. The classification system may utilize real time locating systems, global positioning systems, or other systems now known or later developed in the art to monitor, collect, and store the time-series positional data. The following documents discuss various systems and methods of monitoring, collecting, and storing data: U.S. Pat. No. 6,055,434; U.S. Pat. No. 6,375,612; U.S. Pat. No. 6,569,092; U.S. Pat. No. 6,427,627; U.S. Pat. No. 7,026,941; U.S. Pat. No. 7,543,549; U.S. Patent Publication No. 2002/0010390; U.S. Patent Publication No. 2005/0145187; U.S. Patent Publication No. 2008/0236500; U.S. Patent Publication No. 2008/0312511; U.S. Patent Publication No. 2009/0182207; U.S. Patent Publication No. 2010/0107985; European Patent No. 0 743 043; Great Britain Patent No. 2 353 910; and International Patent Publication No. WO 2008/156416; all of which are hereby incorporated by reference herein in their entireties.

In one configuration, a real-time location monitoring system is utilized to collect time-series positional data of individual animals. However, as noted above, other systems may be used such as global positioning systems. The real-time location monitoring system utilizes radio frequency tags placed on individual animals to wirelessly transmit ultrawideband pulses to receivers mounted at multiple locations around the perimeter of a housing area. The receivers triangulate the animal position and transfer the data to a database that accumulates and stores the time series positional data. Each record in the raw data includes an individual animal identification number, a timestamp (hours, minutes, seconds), and at least a two-dimensional position at the recording time (for example, a X and Y coordinates relative to a known coordinate). A map of the housing area with known boundary and object coordinates is utilized in connection with the animal coordinates to determine the location of individual animals relative to objects of interest in a housing area.

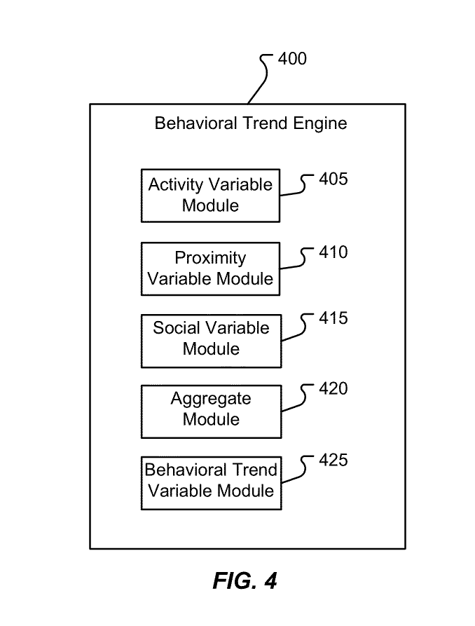

At step 310, a classification system generates behavioral trend data of individual animals. To generate the behavioral trend data, the classification system includes a behavioral trend engine. Referring to FIG. 4, a block diagram of a behavioral trend engine 400 is illustrated. The behavioral trend engine 400 includes an activity variable module 405, a proximity variable module 410, a social variable module 415, an aggregate module 420, and a behavioral trend variable module 425.

The movement patterns of individual animals over time are used to determine the health status of the animals. The behavioral trend engine 400 includes an activity variable module 405 that creates activity variables from the positional data 305. The activity variables generally describe the activity of individual animals over a predefined period of time. Activity variables include variables describing the distance traveled by individual animals and the speed of travel of individual animals.

The activity variable module 405 generally sorts the positional data 305 by individual animal and calculates the time, in seconds, that each individual animal spent at each known set of coordinates. To create a distance traveled variable for each individual animal, the activity variable module 405 compares the X and Y coordinates from consecutive time positional measurements for each individual animal and calculates the distance traveled between the points using the Pythagorean theorem. To create a speed variable for each individual animal, the activity variable module 405 divides the distance traveled between two points by the time lapse between the two locations. The activity variable module 405 may create additional variables from the speed variable such as a traveling speed average, a maximum speed, and a percent of time the animal traveled at greater than a predefined speed, which may be 2 meters per second. The activity variables and associated data are stored in a database for further evaluation.

The location of individual animals relative to areas of interest such as feed, water, shelter, and a boundary of a housing area provide insight into the behavioral changes indicative of disease status. The behavioral trend engine 400 includes a proximity variable module 410 that creates proximity variables from the positional data 305. Proximity variables include variables describing an individual animal's proximity to fixed locations, such as areas of interest, within a housing area. Areas of interest include food, water, shelter, and the periphery of the housing area.

The proximity variable module 410 compares the location data 305 of individual animals to predefined areas of interest within the housing area. The housing area is divided into areas of interest including a feeding area, a shelter area, a water area, and a boundary area, which may be mutually exclusive. The coordinates of the areas of interest are saved in a database and utilized by the proximity variable module 410. In addition, a zone surrounding the areas of interest is calculated based on a predetermined distance from the area of interest. The coordinates of the zone surrounding the areas of interest are saved in a database and utilized by the proximity variable module 410. In one embodiment, specific areas of the housing area can be classified both as directly in the object of interest (e.g. feed area) or within a predetermined zone, for example 1.5 meters, surrounding the area (e.g. feed zone). The zone represents a wider region around the object of interest that indicates an animal is following the group, but not participating in a specific activity.

The proximity variable module 410 compares the coordinate data of individual animals to the coordinates of predefined areas of interest and to the coordinates of predetermined zones within the housing area to calculate the number of times an individual animal entered an area of interest, a zone, or both. For example, the proximity variable module 410 calculates the number of events, or bouts, by using consecutive readings in the same location or zone as a single event, or bout. The proximity variable module 410 generates variables describing the number of events, or bouts, at a feed area, a shelter area, a water area, a boundary area, a feed zone, a shelter zone, a water zone, and a boundary zone, for example. Using the time series positional data 305, the proximity variable module 410 also calculates the average number of events, or bouts, at an area of interest or zone over a designated time period. The proximity variable module 410 generates variables describing the average number of events, or bouts, at a feed area, a shelter area, a water area, a boundary area, a feed zone, a shelter zone, a water zone, and a boundary zone over a designated time period, for example.

Using the time series positional data 305, the proximity variable module 410 calculates the amount of time each individual animal spent in each event, or bout, at an area of interest or zone. The proximity variable module 410 generates variables describing the amount of time each individual animal spent at a feed area, a shelter area, a water area, a boundary area, a feed zone, a shelter zone, a water zone, and a boundary zone, for example. The proximity variable module 410 also calculates the average amount of time each individual animal spent in each event, or bout, at an area of interest or zone over a set period of time. The proximity variable module 410 generates variables describing the average amount of time each individual animal spent at a feed area, a shelter area, a water area, a boundary area, a feed zone, a shelter zone, a water zone, and a boundary zone over a designated time period. The proximity variable module 410 also calculates the percent of time each individual animal spent in an area of interest or zone over a set period of time. The proximity variable module 410 generates variables describing the percent of time each individual animal spent at a feed area, a shelter area, a water area, and a boundary area, a feed zone, a shelter zone, a water zone, and a boundary zone over a set period of time.

Animals are social in nature and the interaction patterns with other individuals within the housing area are indicative of health status. The behavioral trend engine 400 includes a social variable module 415 that creates social variables from the positional data 305. Social variables include variables describing social interactions between individual animals and other animals. Social interactions include the proximity of an individual animal to other animals within a defined area and the amount of time an individual animal spends within a certain proximity to other animals.

The social variable module 415 uses the positional data 305 of individual animals to determine the distance between each animal in the housing area and creates a social variable describing the average distance of the closest animal. The social variable module 415 calculates isolation and social indices for each individual animal in the housing area based on the distance between the individual animal and the remaining animals in the housing area. The social variable module 415 determines the distance to the closest animal and the number of animals within a set distance to calculate the isolation and social indices.

To calculate an isolation index for an individual animal, the social variable module 415 determines the percent of time the individual animal spent with no animals within a set distance, such as one, three, and five meters, of the individual animal. The social variable module 415 creates social variables describing the percent of time spent with no animals within one meter, the percent of time spent with no animals within three meters, the percent of time spent with no animals within five meters, the percent of time with the closest animal at least seven meters away, the percent of time with the closest animal at least ten meters away, and the percent of time with the closest animal at least fifteen meters away, for example.

To calculate a social index for an individual animal, the social variable module 415 determines the percentage of time the individual animal spent with a set number of animals, such as four, seven, or ten animals, within a set distance, such as one, three, and five meters, of the individual animal. The social variable module 415 creates social variables describing the average number of animals within one meter, the average number of animals within three meters, the average number of animals within five meters, the percent of time with four or more animals within three meters, the percent of time with four or more animals within five meters, the percent of time with seven or more animals within three meters, the percent of time with seven or more animals within five meters, the percent of time with ten or more calves within three meters, and the percent of time with ten or more animals within five meters, for example.

The variables associated with individual animals can be aggregated over a predetermined time period, such as hourly, before calculating behavioral trend changes of individual animals over time. Referring to FIG. 4, the behavioral trend engine 400 includes an aggregate module 420 that accounts for circadian movement and activity patterns associated with animal behavior. The aggregate module 420 calculates hourly values for each of the activity, proximity, and social variables created by the activity variable module 405, the proximity variable module 410, and the social variable module 415, respectively. The aggregate module 420 sums the time variables for each hour. For example, the aggregate module 420 sums the amount of time an individual animal spent in an activity or location during any given hour. The aggregate module 420 uses the aggregated time variables to determine time percentages for each hour. For example, the aggregate module 420 determines the percentage of time an individual animal spent in an activity or location during any given hour. The aggregate module 420 also sums the count variables for each hour. For example, the aggregate module 420 sums the events, or bouts, of an activity to create a total number of events, or bouts, during any given hour.

The behavioral trend engine 400 also includes a behavioral trend variable module 425 that performs a number of time series calculations to calculate changes in behavioral trends of individual animals over time and creates corresponding behavioral trend variables. The behavioral trend variable module 425 performs calculations on the variables created by the activity variable module 405, the proximity variable module 410, and the social variable module 415 to calculate behavioral trend variables including moving averages and control limits. The variables may be aggregated by the aggregate module 420 before the behavioral trend variable module 425 calculates the behavioral trend variables. The behavioral trend variables can be calculated over predetermined time periods.

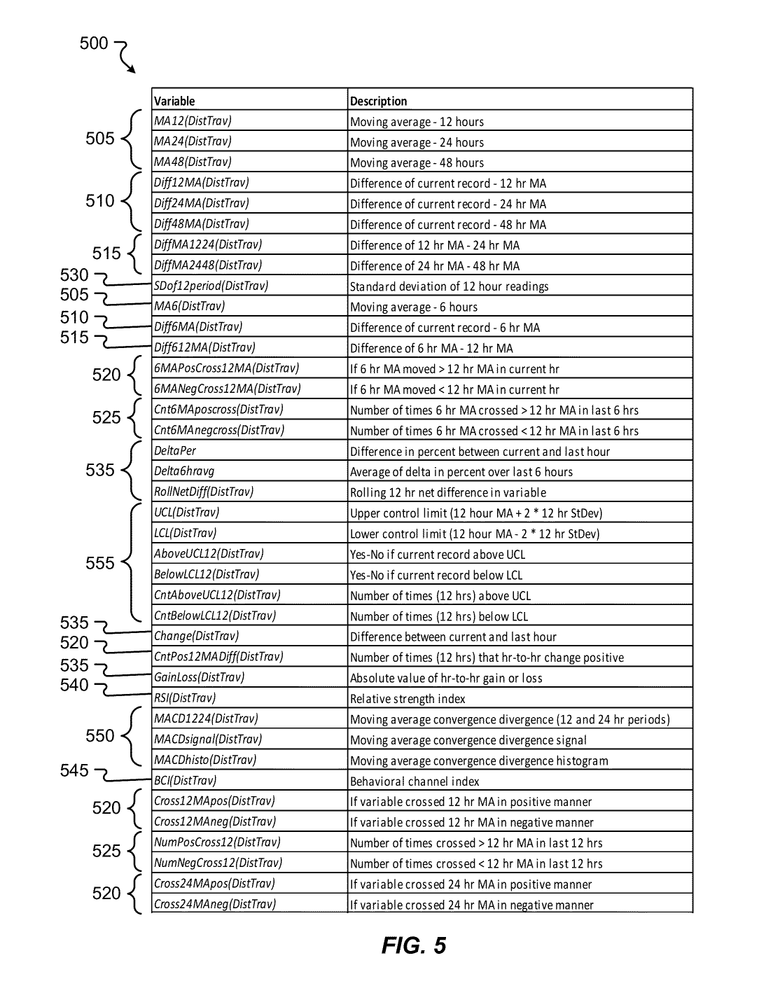

The behavioral trend variable module 425 calculates moving averages for each of the activity, proximity, and social variables over set time periods, such as six, twelve, twenty-four, and forty-eight hour periods. FIG. 5 illustrates a list of behavioral trend variables 500 calculated for a distance traveled activity variable. Moving average variables 505 are calculated over six, twelve, twenty-four, and forty-eight hour periods for the distance traveled activity variable.

The behavioral trend variable module 425 calculates differences between the hourly value for each animal and a moving average. FIG. 5 illustrates moving average difference variables 510 describing the differences calculated between the current record and six, twelve, twenty-four, and forty-eight hour moving averages for the distance traveled activity variable.

The behavioral trend variable module 425 calculates differences among moving averages over each period, such as between a six and twelve hour moving average, a twelve and twenty-four hour moving average, and a twenty-four and forty-eight hour moving average. FIG. 5 illustrates behavioral trend variables 500 that include difference among moving average variables 515 calculated for the distance traveled activity variable.

The behavioral trend variable module 425 compares the value of an individual variable to the value of a moving average and creates additional behavioral trend variables to record the results. For example, the behavioral variable module 425 creates binary variables to indicate if the value of the variable crossed the 12, 24, or 48 hour moving averages in a positive or negative direction during the hour of interest. The behavioral trend variable module 425 also creates count variables to sum the total number of positive and negative crosses within a previous time period, for example 12 hours, and place the count variables in the data set as a rolling average. FIG. 5 illustrates behavioral trend variables 500 that include binary variables 520 and count variables 525.

The behavioral trend variable module 425 also calculates a standard deviation of each variable over a previous time period. FIG. 5 illustrates behavioral trend variables 500 that include a standard deviation variable 530 calculated for the distance traveled of an individual animal over the previous 12 hours. The behavioral variable module 425 calculates the changes in each variable between consecutive hours as a delta, or percentage change, and a rolling average of delta is included in the data set. FIG. 5 illustrates change variables 535 in connection with the distance traveled by an individual animal.

The behavioral trend variable module 425 calculates a relative strength index (RSI) over a set time period, for example 12 hours, to quantify the potential positive or negative moves in the trend. The RSI value is calculated as the average value of positive hour-to-hour moves in the variable divided by the average value of negative hour-to-hour moves in the variable over the 12 hours. FIG. 5 illustrates a RSI variable 540 in connection with the distance traveled by an individual animal.

The behavioral trend variable module 425 calculates a behavioral channel index (BCI) for a set time period. The behavioral trend variable module 425 calculates a 12 hour BCI by dividing the difference between the reading of the hour of interest and the 12-hour average by the absolute value of the mean difference between each of the previous 12 periods and the current 12-hour average. FIG. 5 illustrates a BCI variable 545 in connection with the distance traveled by an individual animal.

The behavioral trend variable module 425 calculates moving average convergence and divergence (MACD) values using predefined time periods, such as twelve and twenty-four hour periods, resulting in a MACD line, a signal line, and a MACD histogram value. FIG. 5 illustrates MACD variables 550 in connection with the distance traveled by an individual animal. The behavioral trend variable module 425 also calculates control lines for predefined time periods based on a set standard of deviation. For example, the behavioral trend variable module 425 calculates upper and lower control lines (UCL, LCL, respectively) for each 12 hour period based on a 2 sigma value (or 2 times the standard deviation of the 12 hour period). The number of times the reading is above or below the UCL or LCL is summarized in a count variable for each time period. FIG. 5 illustrates UCL and LCL variables 555 in connection with the distance traveled by an individual animal.

The behavioral trend variable module 425 calculates the behavioral trend variables described above for each of the activity, proximity, and social variables, each of which can be aggregated over a predefined time period prior to calculating the behavioral trend variables. In one embodiment, the activity variable module 405 includes four activity variables, the proximity variable module 410 includes twenty-two proximity variables, and the social variable module 415 includes fifteen social variables. In this embodiment, the behavioral trend variable module calculates trend variables for each of the activity, proximity, and social variables, resulting in a dataset containing 1640 columns (1599 calculated variables plus the original 41 variables). The final data is placed into a single database and represents the overall behavioral trend data 310 for each individual animal.

At step 315 of FIG. 3, a classification system classifies the respiratory health status of individual animals. To assess the respiratory health of individual animals, the classification system evaluates the activity variables, the proximity variables, the social variables, and/or the behavioral trend variables with a dynamic disease detection algorithm. The dynamic disease detection algorithm classifies individual animals as diseased or healthy over a predetermined time period, such as hourly. The classifications are used to determine the overall respiratory health status of individual animals over a predetermined time window, such as six, twelve, or twenty-four hour time periods.

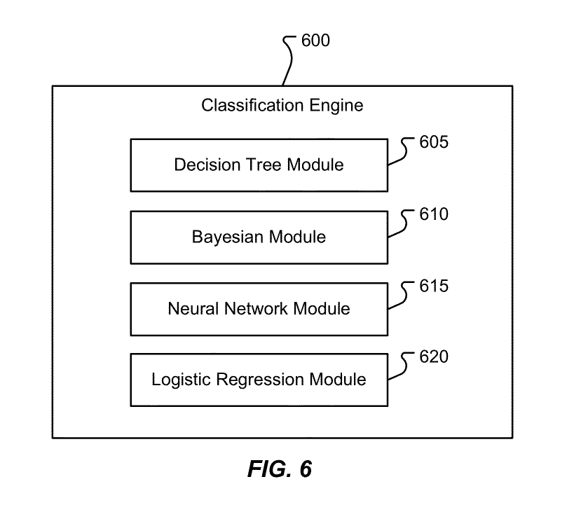

To classify the respiratory health status of an individual animal, the classification system includes a classification engine. Referring to FIG. 6, a block diagram of one embodiment of a classification engine 600 is illustrated. The classification engine 600 includes four classification modules that can be applied to the activity variables, the proximity variables, the social variables, and/or the behavioral trend variables to classify the respiratory health of individual animals. The classification engine 600 includes a decision tree module 605, a Bayesian module 610, a neural network module 615, and a logistic regression module 620. Each module employs a different diagnostic algorithm to classify the behavioral trend data 310. For example, the decision tree module 605 employs a decision tree learning algorithm, the Bayesian module 610 employs a Bayes algorithm, the neural network module 615 employs an artificial neural network algorithm, and the logistic regression module 620 employs a logistic regression algorithm. The classification engine 600 utilizes any combination of modules, and thus algorithms, to classify the health status of individual animals. For example, in one embodiment, the classification engine 600 utilizes at least two diagnostic algorithms to classify the health status of an individual animal. A series of disease detection algorithms can be utilized based on the complexity of disease expression in a population.

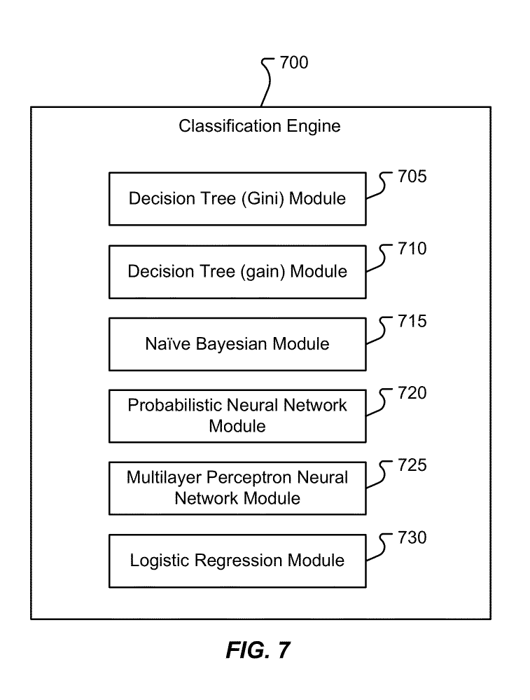

Referring to FIG. 7, a classification engine 700 includes up to six classification modules. The modules include a decision tree (Gini) module 705, a decision tree (gain) module 710, a naïve Bayesian module 715, a probabilistic neural network module 720, a multilayer perceptron neural network module 725, and a logistic regression module 730. Each module employs a different diagnostic algorithm to classify the behavioral trend data 310. For example, the decision tree (Gini) module 705 employs a Gini impurity decision tree algorithm, the decision tree (gain) module 710 employs an information gain decision tree algorithm, the naïve Bayesian module 715 employs a naïve Bayesian classifier algorithm, the probabilistic neural network module 720 employs a probabilistic neural network algorithm based on dynamic decay adjustment, the multilayer perceptron neural network module 725 employs a backwards propagation multilayer perceptron neural network algorithm, and the logistic regression module 730 employs a logistic regression algorithm. The classification engine 700 can utilize any combination of modules, and thus algorithms, to classify the health status of individual animals. For example, in one embodiment, the classification engine 700 utilizes at least two diagnostic algorithms to classify the health status of an individual animal.

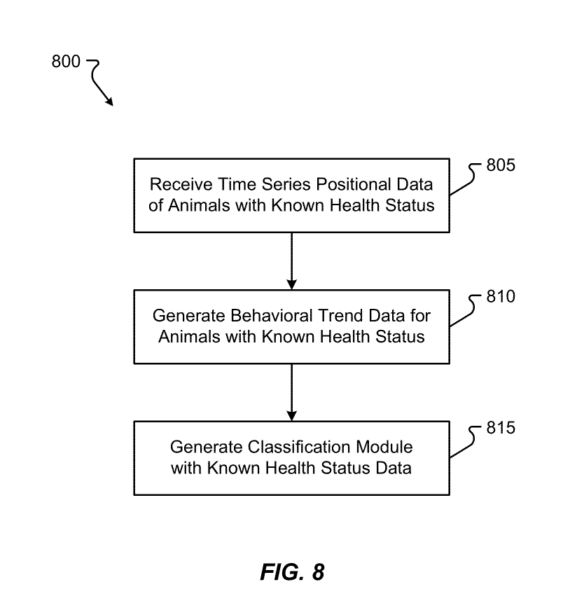

Referring now to FIG. 8, a flowchart 800 illustrating one embodiment of generating a classification module is provided. At step 805, a classification system receives time series positional data of animals with known health statuses, such as healthy or sick. The positional data can be collected in conjunction with monitoring clinical signs and pulmonary pathology. At step 810, a classification system generates behavioral trend data for animals, such as calves, with known health statuses. This process enables the generation of a dataset of behavioral trend data with known health outcomes. At step 815, the behavioral trend data of animals having known health statuses are utilized to generate a classification module.

The historical behavioral and respiratory health data 805, 810 collected on animals of known health statuses are utilized to build a series of predictive classification modules. Each of the modules use the criteria established with the known status individuals to classify the behavioral status of individual animals with unknown statuses as diseased or healthy. The historical data used to generate the classification models can be augmented with subsequently collected information on known outcomes. In one embodiment, information is collected from specific operations and used to create customized models for specific situations. The model generation can be modified to match animals of known demographic status.

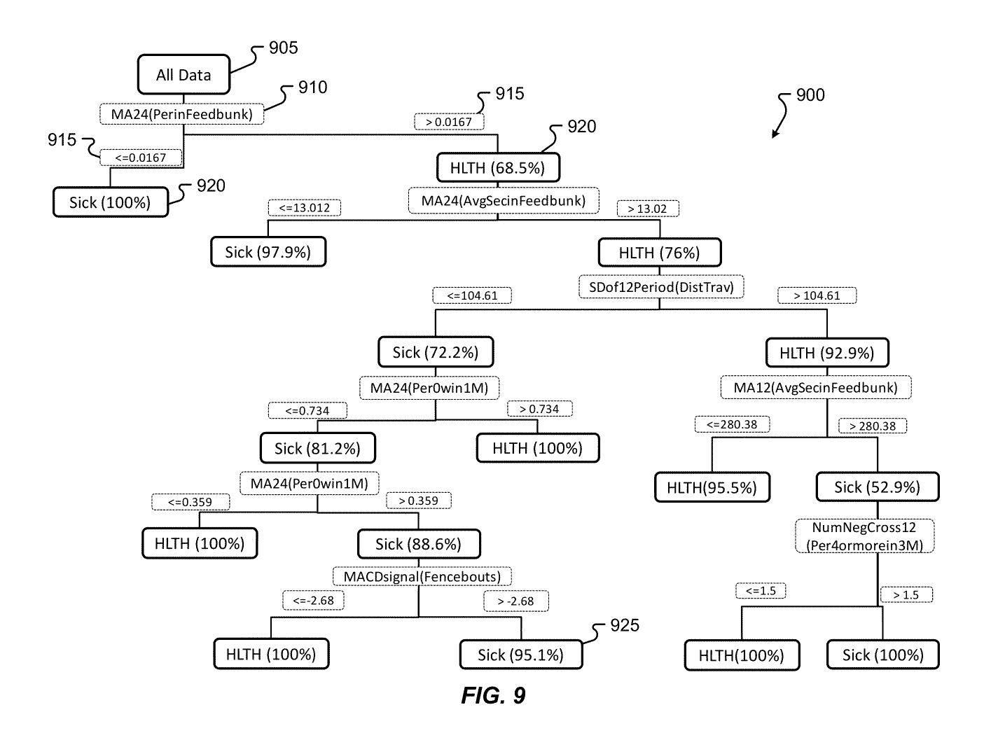

FIG. 9 is a block diagram of one embodiment of a decision tree module 900 utilizing a gain algorithm. Historical behavioral and respiratory health data 805, 810 are used to create a decision tree module utilizing Gini or gain algorithms. Each decision tree utilizes available variables to divide the data into uniform groups based on the target attribute (respiratory health status) and the individual splitting rules. Additional nodes or splits based on proximity, activity, social, and/or behavioral trend variables are generated until the tree is completed. Then the tree is pruned using the method detection limit (MDL) method, and the minimum number of records per node is set at 2.

Referring to FIG. 9, an example decision tree (gain) 900 is displayed. At each level in the tree, animal behavioral data 905, which may be hourly, is split based on the variable with the highest split criteria 910 and the variable splitting value 915, both of which are determined by the decision tree algorithm. The decision tree algorithm then classifies the behavioral data 905 as either sick or healthy and includes the percent of historical records 920 matching the classification.

In the decision tree depicted in FIG. 9, the 24 hour moving average of the percent time spent at the feedbunk 910 was the most informative variable and thus is included at the top level of the decision tree. If an individual animal, such as a calf, has a 24 hour moving average of less than or equal to 1.7 percent at the feedbunk, the behavioral data of the individual animal is classified as sick. As the decision tree progresses to lower levels, further splits are made to classify individual animals as healthy or sick based on the historical data of animals with known health outcomes. Variables can be used more than one time at different split points to create more discrete classification of the animals. Referring to a single node for illustrative purposes, behavioral data in node 925 are classified as sick and there is a 95.1 percent chance that this diagnosis is correct. To be categorized in this node, individual animals met the following criteria: 24 hour moving average percent time in feedbunk greater than 1.7 percent, 24 hour moving average seconds in feedbunk greater than 13.02 seconds, standard deviation of 12 hour distance traveled less than or equal to 104.61, 24 hour moving average of percent of time with zero animals within 1 meter less than or equal to 0.734, and 24 hour moving average of percent time with zero animals within 1 meter greater than 0.359, and the moving average convergence divergence signal related to fence bouts is greater than −2.68.

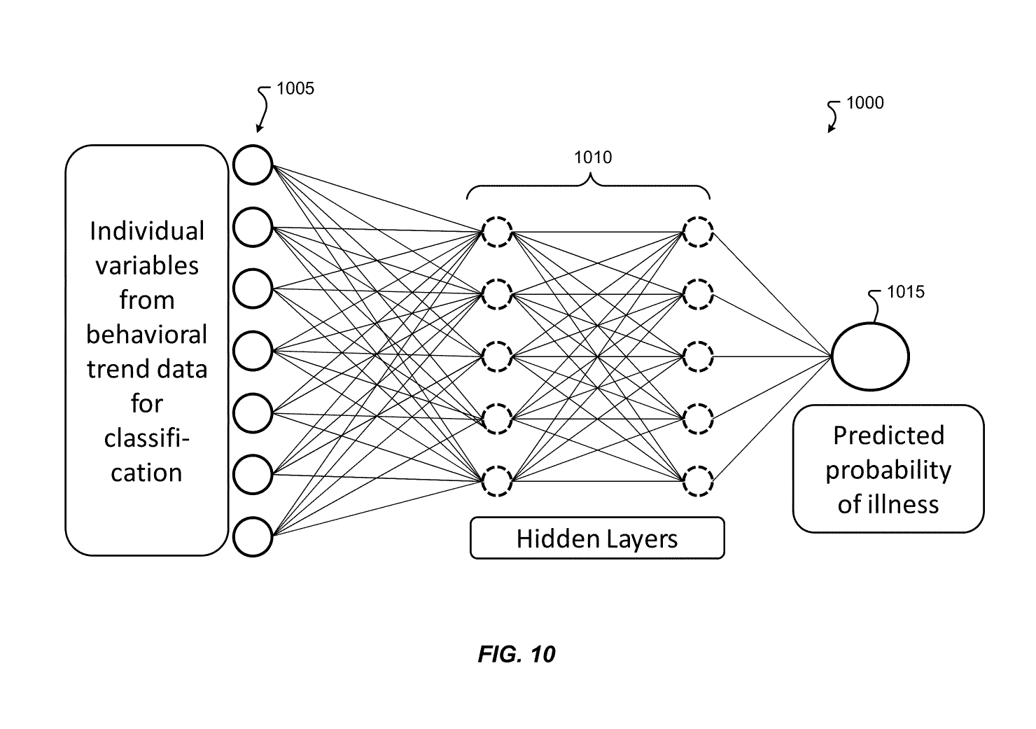

FIG. 10 depicts a block diagram of one embodiment of a neural network module framework 1000. The network 1000 is built using behavioral trend data from animals, such as calves, with known health outcomes. After being built, subsequent animals, such as calves, are classified utilizing the module. Although only seven variables 1005 are illustrated in FIG. 10, some neural network models include over 570 individual variables that are fed into the network 1000. Each variable 1005 is tied to two hidden layers 1010 by a series of equations. The specific equations joining the raw data and the hidden layers is dictated by the type of neural network implemented, which may include probabilistic or backwards propagation multilayer perceptron neural network. The mathematical formulas generated in aggregate by the neural network result in a predicted probability of illness 1015.

Referring back to FIG. 7, the classification engine 700 includes two types of artificial neural networks 720, 725 that are generated using the historical behavioral and respiratory health data 805, 810 of animals with known health statuses. A probabilistic neural network module 720 based on dynamic decay adjustment is created using numeric data. The neural network module 720 employs an algorithm that creates a series of rules defined as high-dimensional Gaussian function that are defined by a center vector and a standard deviation. The selected numeric columns are used to predict the target class (respiratory health status). A backwards propagation multilayer perceptron neural network module 725 also is generated using the data 805, 810. The neural network module 725 classifies the data 305, 310 using numerical columns to predict the target class (respiratory health status).

Still referring to FIG. 7, the classification engine 700 includes a Bayesian module 715. The historical behavioral and respiratory health data 805, 810 are used to generate the Bayesian module 715. For nominal attributes, the frequency of occurrence is used and Gaussian distributions are used for numerical attributes. The prediction is based on the product of the probability per attribute and the probability of the behavioral data, which may be hourly, representing sickness or health (based on known baseline data). The Bayesian module 715 utilizes all available data 305, 310 to predict the respiratory health status of individual animals. The Bayesian module 715 gives no consideration to the underlying relationships between each of the variables included in the dataset.

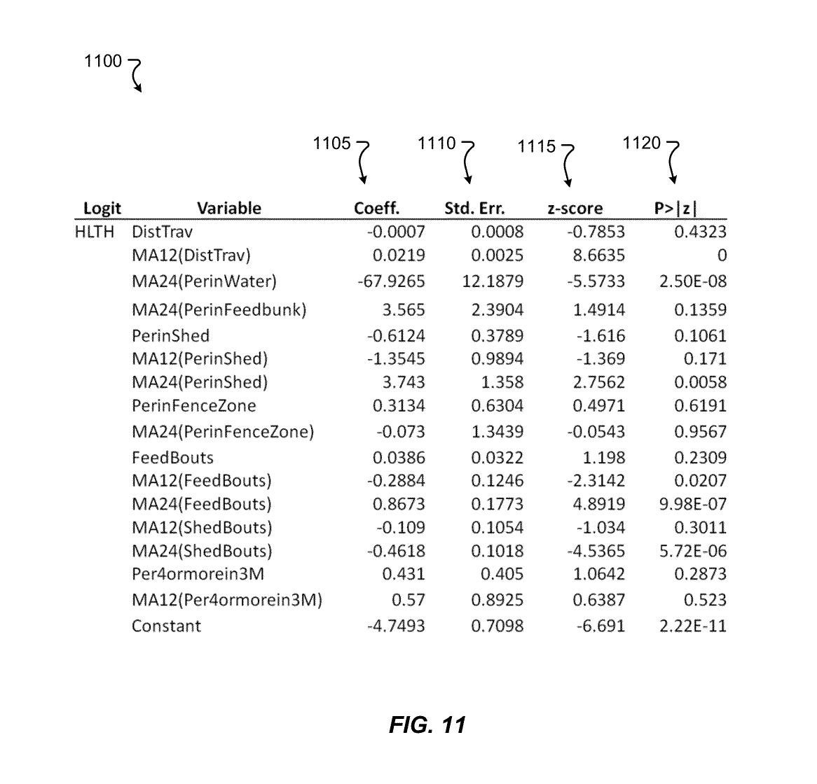

Referring to FIG. 11, the results from a logistic regression model 1100 including coefficients 1105, standard errors 1110, z-scores 1115, and p-values 1120 that have been generated based on data 805, 810 from animals, such as calves, with known health outcomes are illustrated. The results are used in building a logistic regression module 620 or 730 that is used to classify the health status of individual animals with unknown health outcomes.

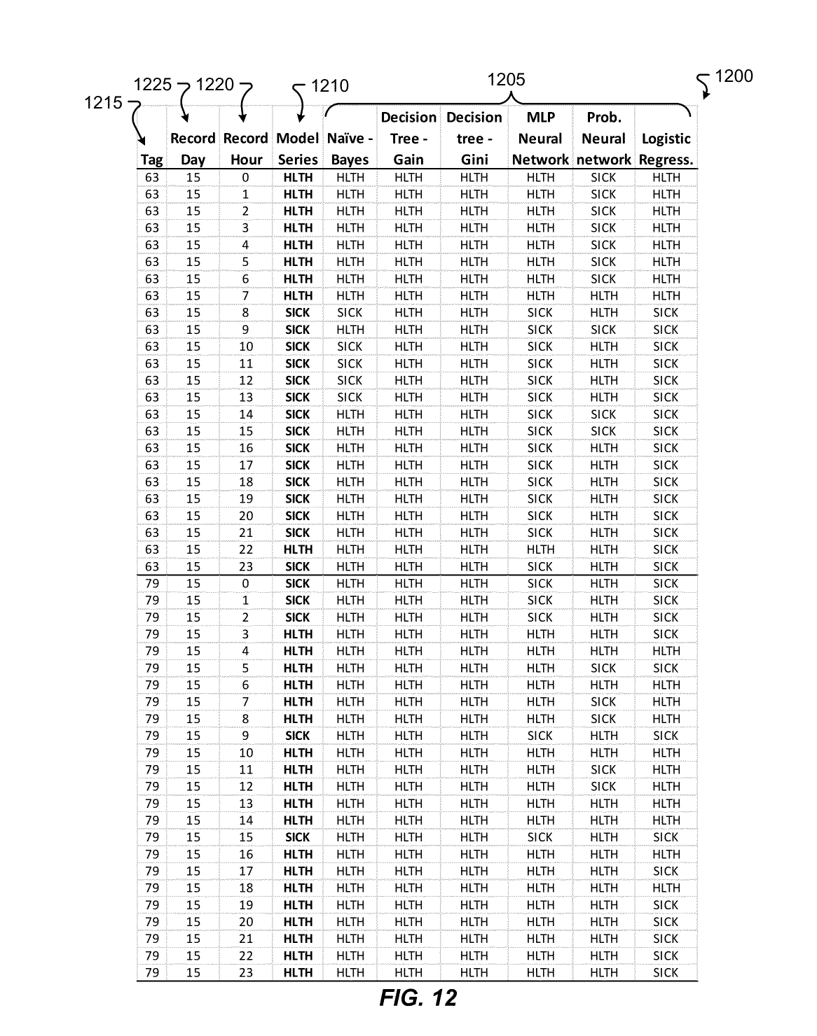

After generation of each of the classification modules, the classification engine 700 classifies the behavioral trend data 310. In one embodiment, the classification engine 700 uses each of the modules to classify the respiratory health status of individual animals for each hour. Referring to FIG. 12, a sample report 1200 illustrates the results from each of the predictive models 1205, including the cumulative results labeled as model series 1210, for two independent animals denoted by tag number 1215 over a 24-hour period 1220. The dataset illustrated by the sample report 1200 represents the cumulative knowledge gained from each of the six decision modules used to classify the current behavioral trend data 310 of two individual animals. Each row in this dataset includes the health state predictions from each of the modules for each hour 1220 of the record day 1225. The table illustrates that the health state predictions of the classification modules 1205 do not always agree. Thus, the classification engine 700 combines data from multiple models 1205 to achieve a single classification 1210 for each hour. In one embodiment, the classification engine 710 classifies the animal as diseased when greater than 50% of the test population was determined to be ill with this specific combination of module classifications. The model series column 1210 represents a classification based on the unique combination of health status classified by each individual model 1205 and validated on cattle with known health outcomes.

Decisions regarding health actions typically are made once or twice per day. Therefore, the hourly health data are aggregated to predetermined time periods, such as twelve hour periods. The hourly health data may be aggregated into one health classification from 5 am to 5 pm and another health classification from 5 pm to 5 am. The number of hours classified as abnormal, or respiratory diseased, is utilized to generate a final actionable respiratory health status of the individual animal (step 320 of FIG. 3). The classification system classifies an animal, such as a calf, as sick if the animal was classified as sick for greater than a predetermined percentage, such as 60 percent, of the classification time period, or seven of twelve hours in the example illustrated in FIG. 12. Referring to FIG. 12, the animal with tag number 63 was classified as SICK 1210 for five of twelve hours 1220 during the first twelve hours in the data. Therefore, the classification system classifies the animal as healthy for this twelve hour period. In the subsequent twelve hour time period, the animal with tag number 63 had eleven of twelve hours classified as SICK 1210. Thus, the animal would be classified as sick for the subsequent twelve hour period. Still referring to FIG. 12, the animal with tag number 79 had less than seven hours classified as diseased for each twelve hour time period. Thus, the animal with tag number 79 would be classified as healthy for both twelve hour periods illustrated in FIG. 12.

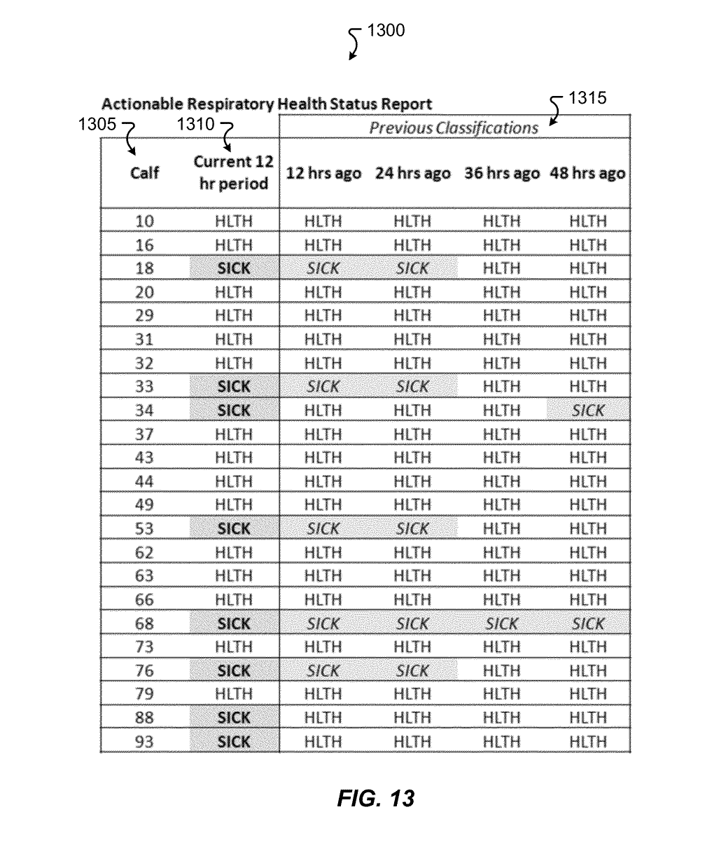

The output of the classification modules is summarized into a single classification for each designated time period and presented to the end user in an actionable respiratory health status report (step 320 of FIG. 3). An example of this report is displayed in FIG. 13. The respiratory health status report 1300 displays the respiratory health status of individual calves 1305 for the current twelve-hour period 1310 and previous periods of time 1305. The report 1300 illustrates how the individual calves 1305 transitioned from a state of health to illness. The current status 1310 of the animal in view of the previous classifications 1315 can be used to make treatment and health intervention decisions. Depending on the preferences of the operation, these interventions may involve further examination of the animal or treatment based on the classification.

The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. Moreover, while flowcharts have been discussed and illustrated in relation to a particular sequence of events, it should be appreciated that changes, additions, and omissions to this sequence can occur without materially affecting the operation of the disclosed embodiments, configuration, and aspects. Furthermore, while various embodiments have been described in detail, it should be understood that modifications and alterations of those embodiments will occur to those skilled in the art. It is to be expressly understood that such modifications and alterations are within the scope and spirit of the claimed subject matter, as set forth in the following claims.

What is claimed is:

1. A method of classifying the respiratory health of an individual animal having an unknown respiratory health status, the method comprising:
receiving time-series positional data of the individual animal;
creating, via a processor, at least one activity variable describing movement of the individual animal, at least one proximity variable describing the proximity of the individual animal to an area of interest, and at least one social variable describing social interactions of the individual animal with other animals;
calculating, via a processor, at least one behavioral trend variable based on the at least one activity variable, the at least one proximity variable, and the at least one social variable for the individual animal;
classifying, via a processor, the respiratory health status of the individual animal based on the output of at least two diagnostic algorithms, each of which receive, as inputs, the at least one activity variable, the at least one proximity variable, the at least one social variable, and the at least one behavioral trend variable for the individual animal; and
generating, via a processor, a report detailing the respiratory health status of the individual animal over a designated time period.

2. The method of claim 1, further comprising:
receiving historical behavioral and respiratory health data of a plurality of animals having a known respiratory health status; and
using the historical behavioral and respiratory health data of the plurality of animals having a known respiratory health status to create the at least two diagnostic algorithms.

3. The method of claim 1, further comprising: aggregating, via a processor, the at least one activity variable, the at least one proximity variable, and the at least one social variable over an hourly time period, and wherein the at least one behavioral trend variable is calculated based on the aggregated variables.

4. The method of claim 1, wherein the at least one activity variable comprises a variable describing the distance traveled by the individual animal and a variable describing the speed of travel by the individual animal.

5. The method of claim 1, wherein the area of interest comprises a feeding area, a shelter area, a water area, and a boundary area.

6. The method of claim 1, wherein the at least one behavioral trend variable comprises a moving average variable, an upper control limit variable, a lower control limit variable, a relative strength index variable, and a behavioral channel index variable.

7. The method of claim 1, wherein the at least two diagnostic algorithms comprise a decision tree algorithm, a naïve Bayesian classification algorithm, a neural network algorithm, and a logistic regression algorithm.

8. The method of claim 1, wherein the designated time period comprises a 12-hour period, and wherein the report includes the health status for the current 12-hour period and at least one previous 12-hour period.

9. The method of claim 1, wherein the report is in the form of at least one of a user interface, a printed report, a text message, or an email message.

10. The method of claim 1, wherein the individual animal is a calf.

11. A non-transitory computer-readable medium containing computer executable instructions, wherein, when executed by a processor, the instructions cause the processor to execute a method of classifying the respiratory health of an individual animal having an unknown respiratory health status, the computer-readable instructions comprising:
instructions to create at least one activity variable describing movement of the individual animal, at least one proximity variable describing the proximity of the individual animal to an area of interest, and at least one social variable describing social interactions of the individual animal with other animals;
instructions to calculate at least one behavioral trend variable based on the at least one activity variable, the at least one proximity variable, and the at least one social variable for the individual animal;
instructions to classify the respiratory health status of the individual animal based on the output of at least two diagnostic algorithms, each of which receive, as inputs, the at least one activity variable, the at least one proximity variable, the at least one social variable, and the at least one behavioral trend variable for the individual animal; and
instructions to generate a report detailing the respiratory health status of the individual animal over a designated time period.

12. The computer-readable medium of claim 11, further comprising instructions to aggregate the at least one activity variable, the at least one proximity variable, and the at least one social variable over an hourly time period, and wherein the at least one behavioral trend variable is calculated based on the aggregated variables.

13. The computer-readable medium of claim 11, wherein the at least one behavioral trend variable comprises a moving average variable, an upper control limit variable, a lower control limit variable, a relative strength index variable, and a behavioral channel index variable.

14. The computer-readable medium of claim 11, wherein the at least two diagnostic algorithms comprise a decision tree algorithm, a naïve Bayesian classification algorithm, a neural network algorithm, and a logistic regression algorithm.

15. The computer-readable medium of claim 11, wherein the report is in the form of at least one of a user interface, a printed report, a text message, or an email message.

16. A system for classifying the respiratory health of an individual animal having an unknown respiratory health status, the system comprising:
a memory;
a processor in connection with the memory, the processor operable to execute software modules, the software modules comprising:
an activity variable module configured to create at least one activity variable describing movement of the individual animal;
a proximity variable module configured to create at least one proximity variable describing the proximity of the individual animal to an area of interest;
a social variable module configured to create at least one social variable describing social interactions of the individual animal with other animals;
a behavioral trend variable module configured to calculate at least one behavioral trend variable based on the at least one activity variable, the at least one proximity variable, and the at least one social variable for the individual animal;
a classification engine configured to classify the respiratory health status of the individual animal based on the output of at least two classification modules, each of which receive, as inputs, the at least one activity variable, the at least one proximity variable, the at least one social variable, and the at least one behavioral trend variable for the individual animal; and
a report module configured to generate a report detailing the respiratory health status of the individual animal over a designated time period.

17. The system of claim 16, further comprising an aggregate module configured to aggregate the at least one activity variable, the at least one proximity variable, and the at least one social variable over an hourly time period, and wherein the at least one behavioral trend variable is calculated based on the aggregated variables.

18. The system of claim 16, wherein the at least one behavioral trend variable comprises a moving average variable, an upper control limit variable, a lower control limit variable, a relative strength index variable, and a behavioral channel index variable.

19. The system of claim 16, wherein the at least two classification modules comprise a decision tree module configured to classify the respiratory health status of the individual animal based on a decision tree algorithm, a Bayesian module configured to classify the respiratory health status of the individual animal based on a naïve Bayesian classification algorithm, a neural network module configured to classify the respiratory health status of the individual animal based on a neural network algorithm, and a logistic regression module configured to classify the respiratory health status of the individual animal based on a logistic regression algorithm.

20. The system of claim 16, wherein the report module is configured to generate a report in the form of at least one of a user interface, a printed report, a text message, or an email message.

21. A method of classifying the respiratory health of an individual animal having an unknown respiratory health status, the method comprising:
receiving time-series positional data of the individual animal;
creating, via a processor, at least one activity variable describing movement of the individual animal and at least one social variable describing social interactions of the individual animal with other animals;
calculating, via a processor, at least one behavioral trend variable based on the at least one activity variable and the at least one social variable for the individual animal;
classifying, via a processor, the respiratory health status of the individual animal based on the output of at least two diagnostic algorithms, each of which receive, as inputs, the at least one activity variable, the at least one social variable, and the at least one behavioral trend variable for the individual animal; and
generating, via a processor, a report detailing the respiratory health status of the individual animal over a designated time period.

22. A method of classifying the respiratory health of an individual animal having an unknown respiratory health status, the method comprising:
receiving time-series positional data of the individual animal;
creating, via a processor, at least one activity variable describing movement of the individual animal and at least one proximity variable describing the proximity of the individual animal to an area of interest;
calculating, via a processor, at least one behavioral trend variable based on the at least one activity variable and the at least one proximity variable;
classifying, via a processor, the respiratory health status of the individual animal based on the output of at least two diagnostic algorithms, each of which receive, as inputs, the at least one activity variable, the at least one proximity variable, and the at least one behavioral trend variable for the individual animal; and
generating, via a processor, a report detailing the respiratory health status of the individual animal over a designated time period.
